(19) United States
(12) Patent Application Publication
(10) Pub. No.: US 2020/0155445 A1
(43) Pub. Date: May 21, 2020

(54) AGED ROSE EXTRACT AND PREPARATION METHOD THEREOF

(71) Applicant: CREALAND CO., LTD, Seongnam-si (KR)

(72) Inventor: SEOK KEE MIN, Wonju-si (KR)

(21) Appl. No.: 16/604,354

(22) PCT Filed: Apr. 9, 2018
(86) PCT No.: PCT/KR2018/004126

§ 371 Date: Oct. 10, 2019

(30) Foreign Application Priority Data
Apr. 10, 2017 (KR) 10-2017-0046279

(51) Int. Cl. A61K 8/9789; A61Q 19/08

(52) U.S. Cl. CPC A61K 8/9789; A61K 2236/333; A61Q 19/08

(57) ABSTRACT

The present invention relates to an aged rose extract and a preparation method therefor and, specifically, provides an aged rose extract having more excellent antioxidant and wrinkle-reducing effects than a conventional rose extract, and provides a method for preparing an aged rose extract, comprising the operations of: (S10) washing rose petals; (S20) preparing an aged rose eluate by mixing the rose petals, which have been washed in operation (S10), with sugars and minerals, and then aging the same; and (S30) extracting the aged rose eluate of operation (S20) with wine.

### TECHNICAL FIELD

The present, invention relates to an aged rose extract and a preparation method therefor and, specifically provides an aged rose extract having more excellent antioxidant and wrinkle-reducing effects than a conventional rose extract, and a method for preparing an aged rose extract, which includes the steps of: washing rose petals (S10); preparing an aged rose eluate by mixing the rose petals, which have been washed in step (S10), with sugars and minerals, and aging the same (S20); and extracting the aged rose eluate of step (S20) with wine (S30).

### BACKGROUND ART

Roses have been widely used for interior decoration or perfumes due to the beautiful appearance and strong fragrance thereof. In particular, Turkey and Bulgaria produce rose oil in mass-production and supply the rose oil as a fragrance for cosmetics all over the world. Recently, the rose is known to be effective for soothing skin and moisturizing skin, so the rose has become an essential ingredient in cosmetics.

The rose is a generic term for plants associated in Dicotyledon order Rosales, family Rosaceae, genus Rosa. The rose is a shrub-type flower tree and there are natural hybrids of wild type and improved breeds. The roses have been cultivated for the purpose of ornament and perfumes due to the beautiful flowers with fragrance and a garden specie grown under improvement is also cultivated. Wild species of the rose are distributed in cold, subtropical, temperate and subtropical regions in the northern hemisphere and about 200 species are known.

Phyto-ingredients such as vitamins A, B, C and E, nicotinic acid, imides, malic acid, organic acids, tannins, flavonoids, and lactones are evenly contained in flowers, leaves, and fruits of the roses, so it is known that the fruit of wild rose was enjoyed as a dessert in the past when the fruit was not common.

Rose petals contain 17 times more vitamin C compared to lemons and 20 times more vitamin A compared to tomatoes. In addition, the petals of roses are known to contain a large amount of polyphenols, carotenoids and anthocyanins, and antioxidant, anti-cancer and anti-inflammatory activities have been reported. Recently, the roses have been reported to be effective in improving depression.

Conventionally, rose extracts have been prepared by hot water extraction using water or solvent extraction using organic solvents such as benzene, methanol, and ether to utilize the roses as a raw material for cosmetics. The conventional hot water extraction is to obtain an extract by filtering a solid component of the rose by heating the rose in water, and since pure water is used as an extraction solvent, the problem caused by the toxicity of the organic solvent does not occur. However, due to the high temperature applied during the extraction, phyto-ingredients such as enzymes and vitamin C contained in the roses are destroyed, the enzyme activity of the roses is inhibited and phyto-ingredients are not obtained sufficiently, so that there is a disadvantage that it is unsatisfied when applied to the skin.

Meanwhile, the extraction method using the organic solvents such as benzene, methanol, hexane, and ether extracts components contained in the roses with organic solvents and then removes the organic solvents. However, although the extraction method can obtain some components and fragrance of the rose simultaneously, it is impossible to completely remove the organic solvents, so there are disadvantages of generating skin troubles such as skin irritation and skin inflammation caused by the organic solvents

Korean Patent No. 10-038909 discloses a conventional technology related to a preparation method of a rose extract as a cosmetic ingredient, in which the rose is washed with purified water, and then the moisture of the rose is dried and the purified water and the preservative are mixed. After that, extraction is performed by using 1,3-butylene glycol, propylene glycol or ethanol as an extraction solvent.

According to the above related art, the skin toxicity caused by the extraction solvent is lowered by using 1,3-butylene glycol, propylene glycol, or ethanol, which are a kind of the cosmetic ingredients, as the extraction solvent of the rose extract, but the active ingredient of the rose is not sufficiently extracted so that a satisfactory effect may not be achieved when applied to the skin.

Korean Patent No. 10-1629031 discloses another conventional technology related to a preparation method of a rose extract, in which the rose is sterilized, inoculated with basidiomycetes for fermentation and then extracted with inorganic or organic solvents. According to the above related art, the rose is extracted after fermenting the rose with basidiomycetes, so that it is possible to prepare an extract having the fragrance of the rose that lasts for a long time. However, there is an inconvenience to perform the sterilization process for fermentation bacteria to mix the rose extract in the cosmetic composition. In addition, some effective ingredients contained in the rose may be destroyed by the high temperature sterilization process, so there is a disadvantage that it is difficult to expect the skin improvement effect such as whitening or wrinkle improvement effect.

Meanwhile, although fermentation and aging are compatibly used with the same concept in the study of natural products, the processes of the fermentation and aging are different from each other. The fermentation is the inoculation of specific microorganisms, such as yeast or bacteria, into natural products to replace natural ingredients with other ingredients through the activity of microorganisms or to obtain the metabolites of microorganisms ingesting natural ingredients. Each fungus produces its own end product, and the fungus's action is visible, so that the fermentation process can be detected. As a representative example of the fermentation, when boiled soybeans are placed on rice straw and left at 40-50° C. for 2-3 days, rice straw subtilis decomposes the soybean protein to produce glutamic acid and sticky viscous substance.

In contrast, aging is the synthesis or change of effective ingredients by the action of an enzyme, which is a kind of a protein such as a gene present in a natural product, by leaving the natural product for a while under certain conditions. The specific ingredients of the natural product may decrease or increase when the natural product is aged and the aging process is often not visible to the naked eye. As an example of the aging, if a refined rice wine is aged for 6 to 12 months after thermal sterilization, stimulant aldehydes are reduced and esters are increased so that the taste is improved. Meanwhile, when the excess sugar is added to the plum at home to make the plum liquid, the fermentation, rather than the aging, may be generally progressed due to the contamination of the microorganism.

All organisms have a variety of enzymes serving as a catalyst for biochemical reactions. Because the enzyme is composed of proteins, it shows all the properties of the protein, and unlike inorganic catalysts, it is strongly influenced by environmental factors such as temperature, pH (hydrogen ion concentration), and protein denaturant. Also, the enzyme may lose activity if the conformation is irreversibly denatured. That is, all enzymes are most activated under certain conditions, and one enzyme represents the catalytic reaction for one chemical reaction or a similar group of chemical reactions, without causing side reactions and producing byproducts.

Meanwhile, Korean Patent No. 10-0389096, Korea Unexamined Patent Publication No. 10-2006-0101096, Korean Unexamined Patent Publication No. 10-2006-0101100, and Korean Patent No. 10-1629031 disclose the technology for preparing the rose extract as a cosmetic ingredient. However, neither of these documents teaches or discloses the aged rose extract and the preparation method thereof according to the present invention.

### DISCLOSURE

### Technical Problem

Accordingly, inventors of the present invention have tried to overcome the problems occurring in the related art in a preparation of an aged rose extract as a cosmetic raw material that phyto-ingredients of a rose are not sufficiently obtained, skin troubles are generated by an extraction solvent, or the phyto-ingredients of the rose are destroyed during the preparation of the extract so that the skin improvement effect is lowered. As a result of diligent efforts to overcome the problems of the related art, the inventors of the present invention have found that the phyto-ingredient of the rose may not be destroyed, the activity of the enzyme contained in the rose is increased to improve the yield of the phyto-ingredients of the rose, and the effect of antioxidant and wrinkle improvement are excellent when extracting the rose with wine after mixing and aging sugars and minerals in the rose.

Therefore, an object of the present invention is to provide an aged rose extract and a preparation method thereof.

### Technical Solution In order to achieve the above object, the present invention provides an aged rose extract and a preparation method thereof.

### Advantageous Effects

According to the aged rose extract and the preparation method thereof of the present invention, the rose is extracted by using natural materials of sugars, minerals, and wines so that skin irritation can be minimized, the phyto-ingredients of the rose can be obtained without destroying the phyto-ingredients as possible, and the effect of improving the antioxidant and skin wrinkles is excellent as compared with the conventional extraction method.

In addition, the aged rose extract and the preparation method thereof according to the present invention have the effect of maximizing the industrial use by applying the aged rose extract, which is excellent in improvement of antioxidant and skin wrinkles, to cosmetic materials.

### DESCRIPTION OF DRAWINGS

FIG. 1 shows collagen biosynthesis acceleration effect according to the mineral content of the aged rose extract of the present invention.

FIG. 2 shows collagen biosynthesis acceleration effect according to an aging period of an aged rose extract of the present invention.

FIG. 3 shows collagen biosynthesis acceleration effect of an aged rose extract according to one embodiment of the present invention and a rose extract of a comparative example.

FIG. 4 shows a free radical scavenging effect of an aged rose extract according to an embodiment of the present invention and a rose extract of a comparative example.

FIG. 5 shows a cell activity effect of an aged rose extract according to an embodiment of the present invention and a rose extract of a comparative example.

### BEST MODE

### Mode for Invention

The present invention provides an aged rose extract and a preparation method thereof.

Hereinafter, the present invention will be described in more detail.

The aged rose extract according to the present invention prepared by mixing sugars and minerals with a rose, and then extracting the rose with wine.

The aged rose extract of the present invention may be easily obtained without adding water or applying heat in the process of obtaining ingredients contained in the rose, and may be easily produced and eluted in a concentrated form without destroying phyto-ingredients of the rose by osmotic pressure caused by sugars.

The minerals and wine may enhance the activity of enzymes contained in the rose, thereby increasing the yield rate of the phyto-ingredients of the rose and improving the antioxidant and anti-wrinkle effect of the aged rose extract.

When extracting the effective ingredients of the rose by fermenting the rose with fungus as disclosed in the related art of Korean Patent No. 10-1629031, the process of sterilizing the fungus is carried out in order to use the aged rose extract as a cosmetic material. In this sterilization process, the phyto-ingredients such as vitamins and polyphenols contained in the rose extract may be destroyed or denatured, thereby reducing the efficacy of the rose extract. Therefore, the present invention provides a method of extracting the phyto-ingredients of the rose by osmotic pressure and aging without inoculation of fungus and without using a fermentation method using the fungus.

The rose of the present invention is a generic term for plants associated in Dicotyledon order Rosales, family Rosaceae, genus Rosa and includes all organs such as flowers, leaves, and stems of wild and improved species, and preferably includes petals of roses belonging to the genus Rosa. More preferably, the rose includes the petals of red rose and white rose.

The sugars of the present invention destroy a cell wall of the rose by osmotic pressure so that the phyto-ingredients contained in the rose may be produced and eluted. The sugars and the rose are mixed at a mixing ratio of 10:90 to 90:10, preferably, 20:80 to 50:50, more preferably, 25:75.

When the mixing ratio of the sugars and the rose exceeds the weight ratio of 20:80 to 50:50, the sugar content is insignificant so that the phyto-ingredient of the rose may not be easily produced and eluted or contaminated by decayed bacteria, In addition, the activity of the enzyme present in the rose may be suppressed due to the excessive sugars, so that the production rate of the phyto-ingredient of the rose may be reduced and the antioxidant and wrinkle improvement effect of the aged rose extract may be lowered. The sugars may include any one or more selected from sugar, honey, glucose, starch syrup, oligosaccharide, fructose, and glucose, preferably, any one or more selected from glucose, sugar and honey.

The mineral of the present invention may Include various complex mineral components such as Ca, Cu, Mg, Mn, Na, K, P, etc., and may serve as a catalyst for accelerating the activity of various enzymes in the rose that activate the phyto-ingredients present in the rose so that the phyto-ingredients of the rose may be more stably produced and eluted. The content of the mineral is preferably 5 to 15% by weight, more preferably, 10 to 15% by weight relative to the total weight of the mixture of sugars and minerals.

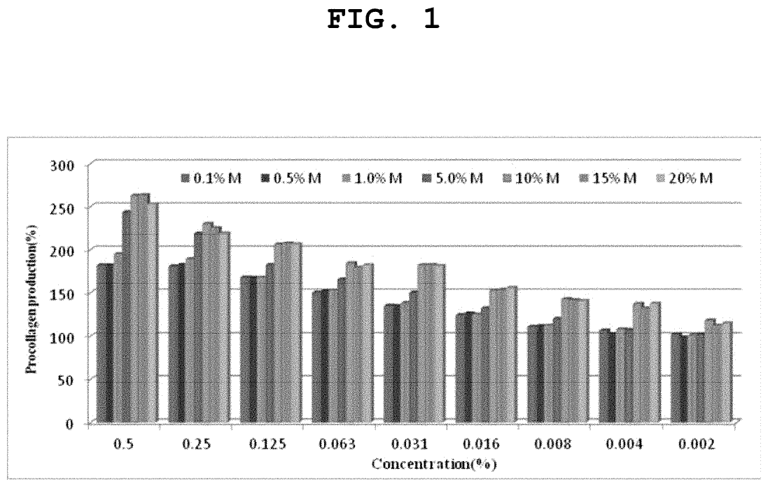

In order to confirm the optimum aging conditions using the minerals, sugars and rose in the present invention, the aging tendency according to the content of used minerals and the concentration of aged rose extract was confirmed. The aging period was 10 weeks, the mineral content was 0.1 to 20% by weight relative to the total weight of the mixture of the rose, sugars and minerals. The results are shown in FIG. 1. As shown in FIG. 1, when the content of the mineral in the aging process IS 5.0 to 15.0% by weight, it was confirmed that the collagen biosynthesis acceleration effect was gradually increased. Meanwhile, according to the result when the concentration of the aged rose extract is 0.25% and 0.5%, it was confirmed that the collagen biosynthesis acceleration effect was slightly decreased when the content of the mineral is 20% by weight compared to when the content of the mineral is 15% by weight. Therefore, it was confirmed that the optimal mineral content based on the efficiency using the rose and sugars is 10-15%.

When the content of the mineral is less than 5% by weight, the mineral content is insignificant so that the effect of enhancing the activity of enzymes in the rose is insignificant, and the phyto-ingredients of the rose may be less produced and eluted so that the antioxidant and anti-wrinkle effect of the aged rose extract may be lowered. When the content of the mineral is more than 15% by weight, the mineral content is excessive, so that the phyto-ingredients of the rose may be less produced and eluted by the osmotic pressure of the sugars and the antioxidant and anti-wrinkle effect of the aged rose extract may be lowered.

The wine refers to an alcoholic beverage, which is obtained by fermenting and aging juice which is generated by crushing grapes, and is divided into red, white, and pink wines depending on the color of the wine or classified into foamable and non-foamable wines depending on the presence of carbon dioxide.

The red wine represents a red color because it is obtained by fermenting a red pigment of a grape shell together with a seed and a shell, and the red wine contains a large amount of minerals, tannins, and vitamins as well as polyphenols such as proanthocyanin, resveratrol, and flavonol. The white and pink wines contain less tannins and anthocyanins than the red wine.

In the present invention, the wine is used as an extraction solvent of the aged rose eluate, but acts as a cofactor for helping the enzyme activity of the rose to improve the antioxidant and anti-wrinkle effect of the aged rose extract. One or more of red wine, white wine, and pink wine may be selected and used, and preferably, one or more of red wine or white wine may be selected and used. The aged rose extract of the present invention may be applied to various fields, and specifically, may be included in a cosmetic composition.

The cosmetic composition may be prepared in various forms, and may be appropriately selected according to the purpose thereof, and specifically, may be prepared in the form of flexible lotion, nourishing lotion, nourishing cream, massage cream, essence, eye cream, cleansing cream, and cleansing foam, cleansing water, pack, spray, powder or lipstick.

In addition, the cosmetic composition may include other components blended with a conventional cosmetic composition as needed. For example, it may include moisturizers, organic or inorganic pigments, ultraviolet absorbers, antioxidants, pH adjusters, alcohols, pigments, perfumes and the like.

Hereinafter, the method for preparing the aged rose extract of the present invention will be described in detail.

The method for preparing the aged rose extract according to the present invention may include washing rose petals (S10); preparing an aged rose eluate by mixing the rose petals, which have been washed in operation (S10), with sugars and minerals, and aging the same (S20); and extracting the aged rose eluate of operation (S20) with wine (S30).

In addition, the method of preparing the aged rose extract of the present invention may further includes aging (S40) for 1 to 4 weeks after performing operation (S30).

First, operation (S10) of washing the rose petals is performed. After removing the foreign substances by washing the rose petals with purified water in operation (S10), only water attached to the surface of the rose petals is removed. In this case, it is desirable not to apply heat so that moisture contained in the rose petals is not lost.

Next, the rose petals washed in operation (S10) is mixed with sugars and minerals and then aged to prepare an aged rose eluate (320) The rose petals and sugars of operation (S10) are mixed at a weight ratio of 50:50 to 80:20 and at the same time, the sugars and the minerals are mixed at 5 to 15% by weight based on the total weight of the mixture of sugars and minerals. In this case, operation (S20) may be carried out for the aging period of 6 to 12 weeks.

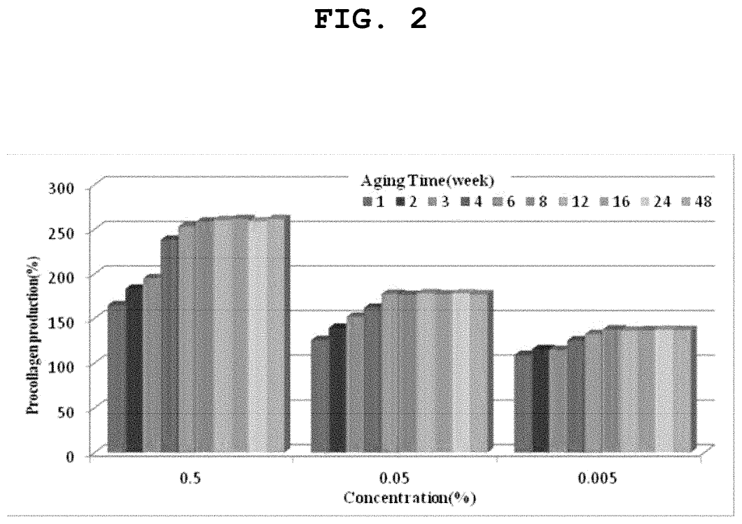

In the present invention, in order to confirm the optimum aging period using minerals, sugars and roses, the content of the mineral was 10-15% based on the total weight of the mixture of sugars and minerals, and the aging state and collagen biosynthesis acceleration tendency of the aged rose extract were configured on a weekly basis. The results are shown in Table 1 and FIG. 2. As a result of confirming the aging trend for 48 weeks, that is, about 1 year, it was confirmed that collagen biosynthesis acceleration effect was increased as the concentration of the aged rose extract was increased regardless of the aging period. Meanwhile, when the aging period was 1 to 6 weeks, it can be seen that the collagen biosynthesis acceleration effect of the aged rose extract was increased with the increase of the aging period, and when the aging period exceeds 12 weeks, the collagen biosynthesis acceleration effect was rarely increased. That is, the time point at which the aged rose extract of the present invention has the optimum effect may be 6 to 12 weeks, and since there is no contamination by the bacteria in the aging process, the aged rose extract was riot denatured even after the aged rose extract was aged for more than 12 weeks.

TABLE 1

| | Aging period | | | | | | | | | |
|---|---|---|---|---|---|---|---|---|---|---|
| | 1 | 2 | 3 | 4 | 6 | 8 | 12 | 16 | 24 | 48 |
| | week | weeks | weeks | weeks | weeks | weeks | weeks | weeks | weeks | weeks |
| Contamination | none | none | none | none: | none | none | none | none | none | none |
| by bacteria | | | | | | | | | | |

When the aging period was 6 to 12 weeks, the antioxidant and anti-wrinkle effect of the aged rose extract was most excellent. When the aging period was more than 12 weeks, the yield rate of the aged rose eluate was not increase significantly compared to the increase of the aging period, and the antioxidant and anti-wrinkle effect of the aged rose extract was rarely improved.

Operation (S20) is desirably carried out at a temperature of 20 to 40° C. When the temperature is 20 to 40° C., the enzyme and phyto-ingredients of the rose petal are produced and eluted without being denatured or destroyed, so that the antioxidant and anti-wrinkle effect of the aged rose extract is the best. If the temperature of operation (S20) is more than 40° C., sugars are denatured during the aging process, so that the phyto-ingredients of the rose petal are not sufficiently produced and eluted, thereby lowering the antioxidant and anti-wrinkle effect of the aged rose extract.

Operation (S30) of extracting the aged rose eluate with wine is performed. Operation (S30) may be carried out by using a pressure filter or a reduced pressure filter, and the reduced pressure filter may be desirably used.

After performing operation (S30), operation (S40) of aging the same for 1 to 4 weeks may be further performed. In this case, operation (S30) may be carried out in a closed container for 1 to 4 weeks. In operation (S40), the wine may act as a cofactor for the enzyme activity of the rose petals, thereby improving the antioxidant and anti-wrinkle effect of the aged rose extract. When the aging period is more than 4 weeks in operation (S40), the wine may be denatured due to the excessive aging, thereby causing the skin problems and lowering the antioxidant and anti-wrinkle effect of the aged rose extract

Hereinafter, the present invention will be described in more detail with reference to preparation examples, examples, comparative examples and experimental examples, These preparation examples, examples, comparative examples and experimental examples are only for illustrating the present invention, and the scope of the present invention is not limited thereto.

### EXAMPLES 1 TO 14

### Preparation of Aged Rose Extract

Using the preparation method described below, the aged rose extract was prepared according to the following rose type, wine type, aging period, and mineral content. In addition, the mineral content was included relative to the total weight of the mixture of sugars and minerals

Operation S10: the rose petals of Table 1 were washed with purified water to remove foreign substances, and only the moisture attached to the surface of the rose petals was removed.

Operation S20: the rose petals of operation S10 and sugars were mixed in a weight ratio of 3:1, and the minerals were mixed at the content of Table 1 based on the total weight of the mixture of sugars and minerals, and then, the aging was performed for the aging period of the following Table 1 at the room temperature of 20 to 30° C. to prepare the aged rose eluate.

Operation S30: the aged rose eluate of operation S20 was extracted with wine of Table 1 by using a reduced pressure filter to prepare the aged rose extract.

TABLE 2

| | | | | Mineral |
|---|---|---|---|---|
| | Rose | Wine | Aging period | content |
| | type | type | of S20 | (weight %) |
| 1Example 1 | Red rose | Red wine | 1 week | 10 |
| | petals | | | |
| Example 2 | Red rose | Red wine | 4 weeks | 10 |
| | petals | | | |
| Example 3 | Red rose | Red wine | 6 weeks | 10 |
| | petals | | | |
| Example 4 | Red rose | Red wine | 8 weeks | 10 |
| | petals | | | |
| Example 5 | Red rose | Red wine | 12 weeks | 10 |
| | petals | | | |
| Example 6 | Red rose | Red wine | 24 weeks | 10 |
| | petals | | | |
| Example 7 | Red rose | Red wine | 48 weeks | 10 |
| | petals | | | |
| Example 8 | Mix red rose | Red wine | 8 weeks | 10 |
| | petals and | | | |
| | white rose | | | |
| | petals at 1:1 | | | |
| | weight ratio | | | |
| Example 9 | White Rose | White wine | 8 weeks | 10 |
| | Petals | | | |
| Example 10 | Red rose | Red wine | 8 weeks | 0.1 |
| | petals | | | |
| Example 11 | Red rose | Red wine | 8 weeks | 1 |
| | petals | | | |
| Example 12 | Red rose | Red wine | 8 weeks | 5 |
| | petals | | | |
| Example 13 | Red rose | Red wine | 8 weeks | 10 |
| | petals | | | |
| Example 14 | Red rose | Red wine | 8 weeks | 20 |
| | petals | | | |

### EXAMPLE 15

### Preparation of Aged Rose Extract

Using the preparation method described below, the aged rose extract was prepared.

Operation S10: the red rose petals were washed with purified water to remove foreign substances, and only the moisture attached to the surface of the red rose petals was removed,

Operation S20: the red rose petals of operation S10 and sugars were mixed in a weight ratio of 3:1, and the minerals were mixed at the content of 10% based on the total weight of the mixture of sugars and minerals, and then, the aging was performed for 4 weeks at the room temperature of 20 to 30° C. to prepare the aged rose eluate. The mineral content was included relative to the total weight of the mixture of sugars and minerals in operation 20.

Operation S30: the aged rose eluate of operation S20 was mixed with the red wine and the same was aged for 4 weeks.

Operation S40: the aged rose eluate of operation S30 was extracted by using a reduced pressure filter to prepare the aged rose extract.

### EXAMPLE 16

### Preparation of Aged Rose Extract

Using the preparation method described below, the aged rose extract was prepared.

Operation S10: the red rose petals were washed with purified water to remove foreign substances, and only the moisture attached to the surface of the red rose petals was removed.

Operation S20: the red rose petals of operation S10 and sugars were mixed in a weight ratio of 3:1, and the minerals were mixed at the content of 10% based on the total weight of the mixture of sugars and minerals, and then, the aging was performed for 8 weeks at the room temperature of 20 to 30° C. to prepare the aged rose eluate. The mineral content was included relative to the total weight of the mixture of sugars and minerals in operation 20.

Operation S30: the aged rose eluate of operation S20 was extracted with the red wine by using a reduced pressure filter.

Operation S40: the extract of operation S30 was aged at the room temperature for 1 week to prepare the aged rose extract.

### COMPARATIVE EXAMPLES 1 TO 4

### Preparation of Rose Extract

### COMPARATIVE EXAMPLE 1

The red rose petals were washed with purified water to remove foreign substances, and only the moisture attached to the surface of the red rose petals was removed. Then, the red rose petals and sugars were mixed at a weight ratio of 3:1, and 10 weight % of minerals based on the total weight of the mixture of the sugars and the minerals was inputted and aged for 8 weeks at the room temperature of 20° C. to 30° C., and the same was extracted by using ethanol as the extraction solvent and a reduced pressure filter, thereby preparing the rose extract of Comparative Example 1.

### COMPARATIVE EXAMPLE 2

The red rose petals were washed with purified water to remove foreign substances, and only the moisture attached to the surface of the red rose petals was removed. Then, the red rose petals and sugars were mixed at a weight ratio of 3:1, and 10 weight % of minerals based on the total weight of the mixture of the sugars and the minerals was inputted and aged for 8 weeks at the room temperature of 20° C. to 30° C., and the same was extracted by using distilled water as the extraction solvent and a reduced pressure filter, thereby preparing the rose extract of Comparative Example 2.

### COMPARATIVE EXAMPLE 3

The red rose petals were washed with purified water, and 100 g of the red rose petals and 10 liters of purified water were inputted in a boiling water distillation device, heated at the temperature of to 80 to 100° C. for 8 hours and filtered, thereby preparing the rose extract.

### COMPARATIVE EXAMPLE 4

The red rose petals were washed with purified water and moisture was removed from the red rose petals. Then, 100 g of the red rose petals were placed in 5 liters of 70% (v/v) ethanol and extracted at the room temperature for 24 hours. The extracted suspension was filtered with a Whatman filter paper, and the filtrate was concentrated under the reduced pressure and vacuum at 50° C., thereby preparing the rose extract.

### EXPERIMENTAL EXAMPLE 1

### Collagen Biosynthesis Acceleration Effect

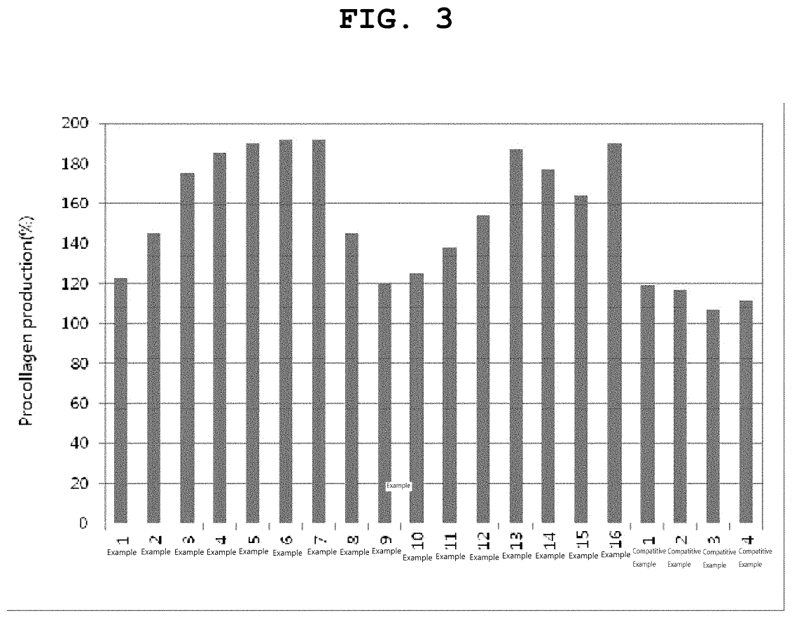

In order to confirm the collagen biosynthesis acceleration effect, of the aged rose extract prepared in Examples 1 to 16, 1×10⁵ human fibroblasts (PromoCell, Germany) were sowed per one well of 24 wells and incubated until they are grown by 90%. After cultivating them in a serum-free DMEM badge for 24 hours, the extracts of Examples and Comparative Examples were dissolved in the serum-free Dulbecco's Modified Eagle's Medium (DMEM) badge, treated at the concentration of 100 ppm, and cultivated in a CO₂ incubator for 24 hours. Supernatants of them were scooped out and checked for increase or decrease of procollagen using the procollagen type (I) Elisa kit (Procollagen type 1), and the results are shown in FIG. 3. The synthetic ability of the procollagen was compared by setting the untreated group to 100.

As shown in FIG. 3, the aged rose extracts of Examples 1 to 16 exhibited excellent collagen biosynthesis acceleration effect compared to the rose extracts of Comparative Examples 1 to 4. That is, it was found that the extract, which is extracted with wine after mixing the rose with sugars and minerals and aging the same, represents the excellent anti-wrinkle effect compared to the extract obtained by using the conventional hot water extraction method and the solvent extraction method.

When comparing Examples 1 to 7, it can be seen that the effect of accelerating the collagen biosynthesis is excellent as the aging period of operation S20 increases, and when the aging period of operation S20 exceeds 8 weeks, the increase in collagen biosynthesis acceleration effect of the aged rose extract was slowed down.

When comparing Example 4 with Comparative Example 1 and Comparative Example 2, it can be seen that the collagen biosynthesis acceleration effect of Example 4 is the most excellent. That is, when taking into consideration that the Example 4 and Comparative Examples 1 and 2 have the same rose type, aging period, and mineral content although extraction was carried out by using wine, ethanol and distilled water, respectively, it may be believed that the wine of Example 4 may maximize and stabilize the production and elution of the phyto-ingredients of the rose while improving the biosynthesis acceleration effect of the aged rose extract.

When comparing Example 4 with Examples 8 and 9, it can be seen that the collagen biosynthesis acceleration effect of Example 4 is the most excellent. In other words, when taking into consideration that the Examples 4, 8 and 9 have the same aging period and mineral content although there are differences in the rose type and the wine type, it may be believed that the aged rose extract obtained by using the red rose petals and the red wine may include a large amount of phyto-ingredients of the rose, which can improve the anti-wrinkle effect, but the aged rose extract obtained by using the white rose and the white wine may include a less amount of phyto-ingredients of the rose, which can improve the anti-wrinkle effect.

### EXPERIMENTAL EXAMPLE 2

### Measurement of Free Radical Scavenging Activity

In order to measure the antioxidant effect of the aged rose extracts of Examples 1 to 16, the free radical scavenging activity was measured.

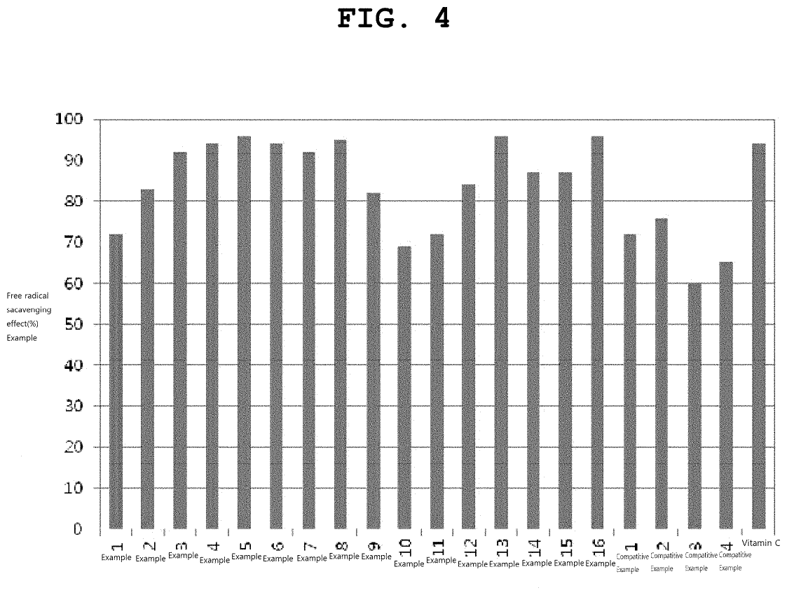

First, after diluting the extracts of the Examples and Comparative Examples to an appropriate concentration in ethanol, 1 M [symbol] is taken and mixed with 1 M [symbol] of 0.1 mM 2,2-diphenyl-1-picryl-hydrazyl radical (DPPH, Sigma) solution. Then, the mixture was left at 37° C. for 15 minutes, and the absorbance was measured at the wavelength of 540 nm using a microplate reader (Thermo max, USA). In the free radical scavenging experiment, the control group was measured in the same manner by adding 1 M [symbol] of DPPH and 1 M [symbol] of ethanol, and the free radical scavenging activity was determined according to Equation 1 below. In this case, vitamin C, which is known to have excellent antioxidant power, was used as a positive control group and the results are shown in FIG. 4.

$$Free radical scavenging activity ( \% ) = \frac{\begin{matrix}Absorbance of control group - \\ absorbance of sample\end{matrix}}{Absorbance of control group} \qquad [ Equation 1 ]$$

As shown in FIG. 4, it can be seen that the aged rose extracts of Examples 1 to 16 represent the superior free radical scavenging effect than the rose extracts of Comparative Examples 3 and 4, and the rose extracts of Comparative Examples 1 to 2 represent the superior free radical scavenging effect than the rose extracts of Comparative Examples 1 and 2. That is, it was found that the extract, which is extracted after mixing the sugars and minerals with the rose petals and acing the same, represents the excellent antioxidant effects than the extract obtained by using the conventional hot water extraction method and the solvent extraction method. Meanwhile, when comparing Example 4 with Comparative Examples 1 and 2, it can be seen that the free radical scavenging effect of Example 4 is the most excellent. In other words, when taking into consideration that the Example4 and

Comparative Examples 1 and 2 have the same rose type, aging period and mineral content although extraction was carried out by using wine, ethanol and distilled water, respectively, it may be believed that the wine of Example 4 may maximize and stabilize the production and elution of the phyto-ingredients of the rose while improving the biosynthesis acceleration effect of the aged rose extract. Meanwhile, when comparing Example 4 with Examples 8 and 9, it can be seen that the free radical scavenging effect of Example 8 is the most excellent. In other words, when taking into consideration that the Examples 1, 8 and Shave the same aging period and mineral content although there are differences in the rose type and the wine type, it may be believed that the aged rose extract, which is extracted by mixing the red rose petals and the white rose petals, may improve the antioxidant effect through the synergistic effect between the phyto-ingredients and enzymes contained in the white rose petals and red rose petals, respectively.

### EXPERIMENTAL EXAMPLE 3

### Evaluation of Cell Activity Using MTT Method

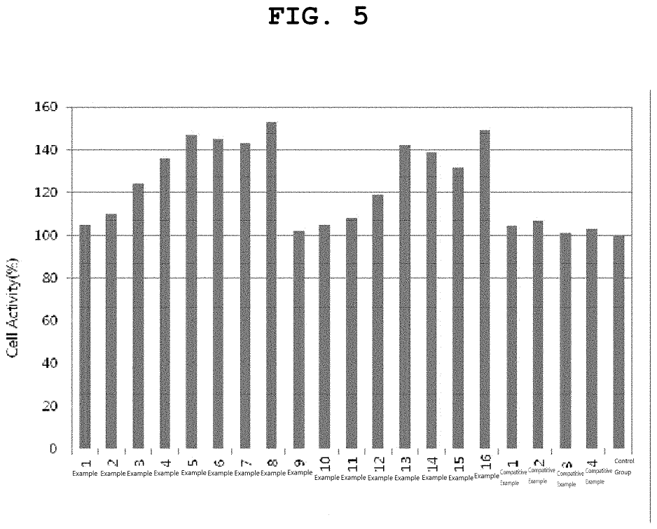

The effect of the aged rose extract prepared in Examples 1 to 16 on the activity of keratinocytes was measured by MTT method. First, 1×10⁵ keratinocytes were inoculated to each well of a 96-well plate per 200 μ [symbol] and cultivated for 24 hours. Then, the extracts of Examples and Comparative Examples were treated at a concentration of 0.1% and cultivated for 48 hours. After removing the culture solution through inhalation, the cells were washed once with PBS, and 100 μ [symbol] of MTT (Methylthiazolyldiphenyl-tetrazolium bromide, Sigma, USA) (0.5 mg/mL) solution was added to the cells and the cells were cultivated at the temperature of 37° C. for 4 hours under the condition of 5% of CO₂. After that, the culture solution was removed through inhalation, and 200 μ [symbol] of DMSO (dimethyl sulfoxide) solution was added and shaken for 10 minutes in a shaker. Then, the absorbance at the wavelength of 540 nm was read with an ELISA reader (Dibiotech, Korea) and the results are shown in FIG. 5. In this case, an untreated group of the aged rose extracts of Examples 1 to 16 was used as a control group.

As shown in FIG. 5, it can be seen that the aged rose extracts of Examples 1 to 16 represent the superior cell activity acceleration effect than the rose extracts of Comparative Examples 3 and 4, and the rose extracts of Comparative Examples 1 to 2 represent the superior cell activity acceleration effect than the rose extracts of Comparative Examples 1 and 2. That is, it was found that the extract, which is extracted after mixing the sugars and minerals with the rose petals and aging the same, represents the excellent cell activity acceleration effect than the extract obtained by using the conventional hot water extraction method and the solvent extraction method.

When comparing Example 4 with Comparative Examples 1 and 2, at can be seen that the cell activity acceleration effect of Example 4 is the most excellent, in other words, when taking into consideration that the Example 4 and Comparative Examples 1 and 2 have the same rose type, aging period and mineral content although extraction was carried out by using wine, ethanol and distilled water, respectively, it may be believed that the wine of Example 4 may maximize and stabilize the production and elution of the phyto-ingredients of the rose while improving the cell activity acceleration effect of the aged rose extract.

When comparing Example 4 with Examples 8 and 9, it can be seen that the cell activity acceleration effect of Example 8 is the most excellent. In other words, when taking into consideration that the Examples 4, 8 and 9 have the same aging period and mineral content although there are differences in the rose type and the wine type, it may be believed that the extract, which is obtained by extracting the red rose petals with the red wine, may represent the superior cell activity acceleration effect than the extract, which is obtained by extracting the white rose petals with the white wine, but the extract, which is extracted by mixing the red rose petals and the white rose petals, may improve the cell activity acceleration effect than the extract, which is obtained by extracting the red rose petals with the red wine, through the synergistic effect between the phyto-ingredients and enzymes contained in the white rose petals and red rose petals, respectively.

### EXPERIMENTAL EXAMPLE 4

### Skin Irritation and Wrinkle Improvement Sensory Evaluation

A cosmetic material containing the extracts prepared in Examples and Comparative Examples was prepared, and the satisfaction level for the skin irritation and wrinkle improvement was evaluated with respect to 80 adult women of 20 to 40 years old in which four test subjects were grouped as one group.

The cosmetic material used herein was an essence formulation, and the composition of which is shown in Table 3 below. In the preparation of the cosmetic materials, first, the ingredients of Table 3 were put into each container in a corresponding content and mixed. Then, the mixture was stirred at 2,000 rpm in the temperature of 50 to 60° C., and cooled at the room temperature to prepare an essence.

The prepared cosmetic materials were used twice a day for 14 days for the test subjects, and the satisfaction level for the skin irritation and wrinkle improvement was progressed by a survey in the form of the sensory evaluation (five-point scale method), in which very good is five points, good is 4 points, normal is 3 points, bad is 2 points, and very bad is 1 point. Evaluation scores are shown in Table 4 below.

TABLE 3

| Raw material | Content (% by weight) |
|---|---|
| Extracts of Examples 1-16 or | 10 |
| Comparative Examples 1-4 | |
| Glycerin | 3 |
| Butylene Glycol | 2 |
| Propylene glycol | 2 |
| Triethanolamine | 0.1 |
| Xanthan Gum | 0.05 |
| 1,2-hexanediol | very small amount |
| Spices | very small amount |
| Purified water | remaining amount |

TABLE 4

| | Skin irritation | Skin wrinkle improvement |
|---|---|---|
| Example 1 | 5 | 1 |
| Example 2 | 5 | 3 |
| Example 3 | 5 | 5 |
| Example 4 | 5 | 5 |
| Example 5 | 5 | 5 |
| Example 6 | 5 | 5 |
| Example 7 | 5 | 4 |
| Example 8 | 5 | 4 |
| Example 9 | 5 | 3 |
| Example 10 | 5 | 3 |
| Example 11 | 5 | 3 |
| Example 12 | 5 | 5 |
| Example 13 | 5 | 5 |
| Example 14 | 5 | 4 |
| Example 15 | 5 | 4 |
| Example 16 | 5 | 5 |
| Comparative Example 1 | 3 | 3 |
| Comparative Example 2 | 5 | 3 |
| Comparative Example 3 | 5 | 2 |
| Comparative Example 4 | 2 | 2 |

As shown in Table 4, it can be seen that the cosmetic materials containing the aged rose extracts of Examples 1 to 16 did not irritate the skin, and Comparative Examples 1 and 4 using ethanol as the extraction solvent induced the skin irritation.

When comparing Examples 1 to 7, it can be seen that the skin wrinkle improvement effect was improved as the aging period is increased, but the skin wrinkle improvement effect of Example 7 having the aging period of 48 weeks was lower than that of Example 6. That is, when taking into consideration the results of Experimental Examples 2 and 3, it may be believed that Example 7 represents a lower level of satisfaction for the skin wrinkle improvement because Example has the free radical scavenging effect and cell activity acceleration effect lower than those of Example 6.

When comparing Example 4 with Comparative Example 1 and Comparative Example 2, it can be seen that. The level of satisfaction for the skin irritation and wrinkle improvement of Example 4 is the most excellent.

When taking into consideration that the Example 4 and Comparative Examples 1 and 2 have the same rose type, aging period and mineral content although extraction was carried out by using wine, ethanol and distilled water, respective it can be seen that Comparative

Example 1 represents a low level of satisfaction for the skin irritation because ethanol used as the extraction solvent causes the skin irritation and Comparative Example 2 does not cause the skin irritation because it uses the distilled water as the extraction solvent.

When comparing Comparative Examples 1 to 4, it can be seen that the rose extracts of Comparative Examples 1 to 2 represent a higher level of satisfaction for the skin wrinkle improvement than the Comparative Examples 3 to 4. That is, it was found that the extract, which is extracted after mixing the sugars and minerals with the rose petals and aging the same, represents the superior skin wrinkle improvement effect than the extract obtained by using the conventional hot water extraction method and the solvent extraction method

1. A method for preparing an aged rose extract, the method comprising:
washing rose petals (S10);
preparing an aged rose eluate by mixing the rose petals, which have been washed in operation (S10), with sugars and minerals and aging the same (S20); and
extracting the aged rose eluate of operation (S20) with wine (S30).

2. The method of claim 1, wherein the sugars and rose petals are mixed in a weight ratio of 20:80 to 50:50.

3. The method of claim 1, wherein the minerals are mixed at 5% to 15% by weight based on a total weight of a mixture of the sugars and minerals.

4. The method of claim 1, wherein an aging period in operation (S20) is 6 weeks to 12 weeks.

5. The method of claim 1, wherein the rose petals are red rose petals, and the wine is a red wine.

6. The method of claim 1, wherein the rose petals are used by mixing red rose petals and white rose petals in a 1:1 weight ratio, and the wine is a red wine.

7. The method of claim 1, wherein further comprising ripening the aged rose extract, which is extracted through operation (S30), for 1 week to weeks (S40).

8. An aged rose extract prepared by the method of claim 4.

9. cosmetic composition comprising an aged rose extract prepared according claim 8.

10. An aged rose extract prepared by the method of claim 3.

11. An aged rose extract prepared the method of claim 2.

12. An aged rose extract prepared by the method of claim 1.
